(19) United States
(12) Patent Application Publication
(10) Pub. No.: US 2022/0257689 A1
(43) Pub. Date: Aug. 18, 2022

(54) CHEWING GUM COMPOSITIONS FOR THE TREATMENT OF MENSTRUAL PAIN

(71) Applicant: ADVANCED FEMALE TECHNOLOGIES LLC, New York, NY (US)

(72) Inventor: Albert Feinstein, New York, NY (US)

(21) Appl. No.: 17/627,493

(22) PCT Filed: Jul. 16, 2020
(86) PCT No.: PCT/US2020/042364

§ 371 Date: Jan. 14, 2022

Related U.S. Application Data
(60) Provisional application No. 62/874,767, filed on Jul. 16, 2019.
(60) Provisional application No. 62/888,006, filed on Aug. 16, 2019.

(51) Int. Cl. A61K 36/71; A61K 36/185; A61K 36/8945; A61K 9/68; A61K 47/46; A61K 47/22; A61K 31/525; A61K 31/355; A61K 33/04; A61K 33/30; A61K 33/24; A61K 31/714; A61K 33/06; A61K 31/519; A61K 31/197; A61K 33/02; A61K 31/704; A61K 31/05; A61K 31/352; A61K 31/353

(52) U.S. Cl. CPC A61K 36/71; A61K 31/353; A61K 36/8945; A61K 9/0058; A61K 47/46; A61K 47/22; A61K 31/525; A61K 31/355; A61K 33/04; A61K 33/30; A61K 33/24; A61K 31/714; A61K 33/06; A61K 31/519; A61K 31/197; A61K 33/02; A61K 31/704; A61K 31/05; A61K 31/352; A61K 36/185

(57) ABSTRACT

This disclosure provides a chewing gum composition comprising an herbal extract and a gum base. The disclosure also provides methods of treating symptoms associated with menopause comprising administering to a subject in need thereof, a gum-based composition for chewing, the composition comprising an herbal extract and a gum base.

### CROSS-REFERENCE TO RELATED APPLICATION

This application claims the benefit of U.S. Provisional Application No. 62/874,767, filed Jul. 16, 2019, and U.S. Provisional Application No. 62/888,006, filed Aug. 16, 2019, both of which are incorporated herein by reference in their entirety.

### FIELD OF THE INVENTION

This disclosure provides a chewing gum composition comprising black cohosh extract and methods of reducing or alleviating symptoms associated with menstruation.

### BACKGROUND

Menstruation is associated with physical symptoms that can include abdominal pain, back pain, pelvic pain, abdominal distension, diarrhea, and constipation. Typical treatment for menstrual pain is symptomatic relief of pain and cramping with combinations of analgesic (including prostaglandin synthetase inhibitors such as aspirin, acetaminophen and ibuprofen), diuretics, antihistamines and antispasmodics. These compositions have been administered in solid form, such as tablets and capsules, and in liquid solutions for treating pain symptoms, such as menstrual cramps and muscle cramps. These compositions commonly include various brands of aspirin, acetaminophen and ibuprofen, which typically provide relief in the form of reduced discomfort from pain and muscle cramping symptoms twenty to forty minutes after ingestion.

Black cohosh (Actaea racemosa or Cimicifuga racemosa) is a perennial herb traditionally used to treat musculoskeletal pain, fever, cough, pneumonia, sluggish labor, and menstrual irregularities.

### SUMMARY OF THE INVENTION

In one aspect, the present disclosure provides a chewing gum composition comprising black cohosh extract and a gum base. In embodiments, the composition further comprises damiana leaf extract and/or wild yam extract. In embodiments, the gum base comprises Couma macrocarpa, glycerol esters of gum, microcrystalline wax, lecithin and calcium carbonate.

In embodiments, the chewing cum compositions further comprise a cannabis extract. In embodiments, the chewing cum compositions further comprise one or more cannabinoids. In embodiments, the one or more cannabinoids include, but are not limited to, cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) and combinations thereof. In embodiments, the one or more cannabinoids is or comprises CBD. In embodiments, the one or more cannabinoids does not include CBD, or includes less than 5%, less than 2%, less than 1%, or less than 0.5% CBD.

In embodiments, the chewing gum composition further comprises one or more sugar alcohols. In further embodiments, the sugar alcohol is maltitol, sorbitol, isomalt, and/or xylitol. The chewing gum compositions may also contain one or more additional sweeteners. In embodiments, the additional sweeteners are selected from a sugar, a high intensity sweetener, a sugar substitute, or a combination thereof. In a further embodiment, the sugar substitute is Stevia.

The chewing gum compositions may also contain one or more flavoring agents, and one or more lubricants and powder flow agents. In embodiments, the flavoring agent is a mint flavoring including, e.g., peppermint oil, oil of wintergreen or spearmint oil. In embodiments, the lubricants and powder flow agents are magnesium stearate, and silicon dioxide.

In embodiments, the chewing gum composition comprises one or more sugar alcohols, a gum base, one or more flavoring agents, one or more sweeteners, an herbal extract, and tableting excipients. In further embodiments, the composition comprises about 45-65% sugar alcohols, about 15-35% gum base, about 2-15% flavoring agents and/or sweeteners, about 2-10% herbal extract and about 1-10% tableting excipients. In embodiments, the composition comprises about 55% sugar alcohols, about 27% gum base, about 9% flavoring agents and/or sweeteners, about 5% herbal extract and about 3% tableting excipients.

Thus, in embodiments, the chewing gum composition, in addition to the black cohosh extract and the gum base, comprises maltitol, sorbitol, isomalt, xylitol, Stevia, a flavoring agent, magnesium stearate, and silicon dioxide. In embodiments, the composition further comprises Damiana leaf extract and/or Wild Yam extract. In further embodiments, the chewing gum composition may include one or more of chromium picolinate, calcium disuccinate, ammonium succinate, zinc difumarate hydrate, tocopherol acetate, glycine, triterpene glycosides, folic acid, vitamin B12, vitamin E, vitamin B6, riboflavin and selenium.

In embodiments, the chewing gum compositions described herein may be manufactured by a method comprising the steps of:
(i) placing a gum base in a container with a sugar alcohol and heating to an internally-measured temperature of between 140-160° F. to melt the gum base;
(ii) combining the herbal extract, sweeteners and flavoring agents, and mixing;
(iii) adding the melted gum base mixture to the mixture from step (ii) and mixing;
(iv) conditioning the mixture from step (iii) for at least about 6 hours at a temperature less than or equal to 75° F. and a relative humidity of 60%;
(v) milling to form particulates;
(vi) mixing the particulates of step (v) with a lubricant; and
(vii) tableting the mixture from step (vi).

In another aspect, the disclosure provides a method of treating symptoms associated with menstruation comprising administering to a subject in need thereof, a gum-based composition for chewing as described in the previous paragraphs and/or as described herein. In embodiments, the one or more symptoms associated with menstruation selected from abdominal pain, back pain, pelvic pain, abdominal distension, diarrhea, and constipation.

### DETAILED DESCRIPTION OF THE INVENTION

Black cohosh extract, is typically administered in the form of tablets, capsules or liquid extract. However such orally administered dosage forms are unable to provide fast relief for reducing or treating symptoms such as pain associated with menstruation. There is a need in the art for a composition that can be used to treat or reduce the symptoms of menstruation such as abdominal pain and cramping where the absorption of the active ingredient is faster than conventional ingestible orally-delivered products such as capsules, pills or liquid.

The present disclosure provides a chewing gum composition comprising a gum base and an herbal extract including black cohosh extract, damiana leaf extract and/or wild yam extract. The chewing gum composition described herein provides a fast release and absorption of active agents from the herbal extract that are effective for the treatment of, or reduction of the discomfort associated with, symptoms of menstruation, including abdominal pain, back pain, pelvic pain, abdominal distension, diarrhea, and constipation. The chewing gum compositions disclosed herein provide a faster onset of action than orally administered dosage forms, which can take up to 40 minutes to absorb through the digestive tract.

Due to the high density of blood vessels in the buccal mucosa, buccally delivered active agents derived from black cohosh extract, damiana leaf extract and/or wild yam extract can gain direct access to the systemic circulation, avoid first-pass hepatic metabolism, and are not exposed to the acidic environment of the gastrointestinal tract. In addition, the buccal mucosa has low enzymatic activity relative to the nasal and rectal routes of administration. Thus, the potential for inactivation of the buccally-delivered extracts due to biochemical degradation is less rapid and extensive than other administration routes.

Herbal Extract

The chewing gum composition of the present disclosure comprise one or more herbal extracts. The term “herbal extract” as used herein is a botanical extract including extract of plant material where the plant is an herb, as well as other plants. The herbal extracts may be derived from any useful part of the plant, including but not limited to leaf, stem, bark, flower, fruit, seed, root, or the entire plaint, by any of several methods of extraction known in the art.

The term “extract” as used herein is a preparation containing one or more active ingredients derived from plant material. Extraction methods include, for example, cold extraction techniques, soxhlet extraction, decoction, supercritical extraction, microwave extraction, and/or extraction using one or more extraction solvents including, but not limited to, ethanol, methanol, acetone, acetonitrile, chloroform, water, hydroalcoholic mixture, and isopropyl alcohol.

Black Cohosh Extract

Black cohosh (Actaea racemosa or Cimicifucra racemose) is a member of the buttercup family (Family Ranunculaceae). It grows in open woods and at the edges of dense forests from Ontario to Tennessee and as far west as Missouri. It is a tall, leafy, perennial herb with a long wand-like raceme of white flowers. The subterranean portion develops as a thick, knotted rhizome system. The roots/rhizomes have been used traditionally by Native Americans to treat colds, rheumatism, and a variety of conditions related to women's health. Black cohosh has traditionally been used to treat conditions including inflammation, dysmenorrhea, cough, diarrhea, rheumatism, arthritis and muscle pain. It has also been used as a sedative and muscle relaxant.

Classes of compounds present in black cohosh extract include triterpene glycosides (actein, cimicifugoside, cimigoside, deoxyacetylacetol, racemoside and 23-epi-26-deoxyactein (previously known as 27-deoxyactein)), phenolic acids (hydroxycinnamic acids, caffeic acid, ferulic acid, and isoferulic acid) and alkaloids (cimipronidine, cyclocimipronidine, and dopargine), salicylic acid, tannins, resin, phytosterols, fatty acids starch and sugars. See Nikolie D., et al. (2015) Nitrogen-Containing Constituents of Black Cohosh: Chemistry, Structure Elucidation, and Biological Activities. In: Jetter R. (eds) The Formation, Structure and Activity of Phytochemicals. Recent Advances in Phytochemistry, vol. 45, Springer, and Black Cohosh Clinical Overview, ABC Clinical Guide to Herbs (2002), pp. 13-22, incorporated herein by reference. Black Cohosh also contains isoflavones including formononetin, however certain preparations such as Remifemin® do not appear to contain appreciable levels of the flavonoids. Black Cohosh Clinical Overview, ABC Clinical Guide to Herbs (2002) at p. 17. Notably, ferulic acid and iso-ferulic acid are two substances found in black cohosh extract that may have anti-inflammatory properties.

Extract of black cohosh may be obtained from the dried rhizome and roots of the plant (Foster S. Black Cohosh: Cimicifuga racemosa—A Literature Review. HerbalGram 1999; 45:35-50; McGuffin M, Kartesz J, Leung A, Tucker A. American Herbal Products Association's Herbs of Commerce, 2nd ed. American Herbal Products Association; 2000).

Several preparations of black cohosh have been described. For example a dried rhizome and root preparation has been described by Bradley P (ed.). British Herbal Compendium Vol. 1. Exeter, UK: British Herbal Medicine Association; 1992:34-6. A decoction may be prepared by adding black cohosh (cut, dried root) to boiling water and simmering for 10-15 minutes. Other preparation include a fluid extract: 1:1 (g/ml) in 90% alcohol. Karnick C., Pharmacopoeial Standards of Herbal Plants, Delhi, India: Srit Satguru Publications; 1994; 1,2:61-2, 13; Newall C, Anderson L, & Phillipson J. Herbal Medicines: A Guide for Health-Care Professionals. London: The Pharmaceutical Press; 1996; Lust J. The Herb Book. New York, N.Y.: Bantam Books; 1974; 124-5, each incorporated by reference.

Some commercial black cohosh extracts have been standardized based upon triterpene glycoside content. Remifemin®, a standardized oral formulation used in black cohosh clinical studies, contains 20 mg of black cohosh extract standardized to 1 mg triterpene glycosides (calculated as 27-deoxyactein) per tablet or twenty drops.

Damiana Leaf Extract

Damiana leaf (Turnera diffusa) is a wild shrub that grows in Mexico, Central America, and the West Indies. Damian leaf is used as a remedy for various diseases in the traditional medicine of Latin America. Damiana is used for its tonic action on the central nervous and hormonal system. Traditionally it has been used as an aphrodisiac, and to treat dyspepsia, diarrhea, and constipation and to improve symptoms of menopause and premenstrual syndrome (PMS). Damiana extract contains a volatile oil containing pinene, cineol, cymol, arbutin, cymene, cadinene, copaenen and thymol, and also contains alkaloids, flavonoids, cyanogenic glycoside, tannins and resin. See U.S. Pat. No. 6,579,543. It has been reported that the most abundant constituents of damiana extract include caryophyllene oxide, caryophyllene, delta-cadinene, elemene and 1,8-cineol. Alcaraz-Melendez, L et al. Analysis of essential oils from wild and micropropagated plants of damiana (Turnera diffusa). Fitoterapia 2004; 75(7-8):696-701, incorporated herein by reference. Another report identified the following flavonoids and a mixture of two compounds, which were identified as naringenin (1), apigenin 7-O-(6″-O-p-E-coumaroyl)-glucoside (2), apigenin 7-O-(6″-O-p-Z-coumaroyl)-glucoside (3), apigenin 7-O-(4″-O-p-E-coumaroyl)-glucoside (4), acacetin (5), genkwanin (6), velutin (7), gonzalitosin I (8), and acacetin 7-O-methyl ether (9). See Willer, J. et al. Cytotoxic Properties of Damiana (Turnera diffusa) Extracts and Constituents and A Validated Quantitative UHPLC-DAD Assay, Molecules (2019), 24(5): 855, incorporated herein by reference.

The Damiana extract is typically taken from the leaf of the plant. Willer, J. et al. describe several methods for preparation of damiana leaf extract (incorporated herein by reference). In the first method, dried herb (1.00 kg) was ground and extracted five times with 2 liters of acetone 70%. The solvent was evaporated under reduced pressure to afford 85 g of crude extract. For isolation, the crude extract was repeatedly partitioned between ethyl acetate and water and the ethyl acetate layer was evaporated to dryness, yielding 32.6 g. Subsequently, this procedure was repeated with butanol (yielding 14.6 g). After acidification of the water layer with 2.5 mL formic acid, the solution was again extracted with butanol (yielding 5.40 g) and the aqueous layer was then evaporated to dryness, yielding 32.4 g. In a second approach, 5.0 g of crude extract were suspended in water and extracted with n-hexane. The fraction was evaporated to dryness. Other extraction methods may utilize solvent extraction with ethanol and water (e.g., about 40% ethanol) from macerated plant material.

Wild Yam Extract

Wild yam refers to species in the genus Dioscorea (Dioscorea polystachya, Dioscorea oppositifolia, Discorea opposita thunb, Dioscorea villosa, Dioscorea alata). Wild yam was traditionally used for its antispasmodic and anti-inflammatory properties to treat menopausal symptoms, gastrointestinal ailments, muscle spasm, asthma, joint pain, and rheumatoid arthritis. Lima C M, et al. Bioassay-guided evaluation of Dioscorea villosa—an acute and subchronic toxicity, antinociceptive and anti-inflammatory approach. BMC Complement Altern Med. Jul. 28 2013; 13(1):195. Wild yam contains compounds including flavonoids, steroidal saponins (dioscin and diosgenin), phytosterols (beta-sitosterol), alkaloids (dioscorin), tannins, starch, mucins, amylase, amino acids (arginine, glutamine, leucine, tyrosine), chlorine, calcium, chromium, copper, iron and vitamin C. Diosgenin, an active ingredient in wild yam, was shown to have estrogenic and progestogenic effects in certain model systems. Park M K, Kwon H Y, Ahn W S, et al. Estrogen activities and the cellular effects of natural progesterone from wild yam extract in mcf-7 human breast cancer cells. Am J Chin Med. 2009; 37(1):159-167. The rhizome and root of wild yam may be extracted by methods known in the art.

Cannabis Extract

In embodiments, the chewing gum compositions disclosed herein comprise a cannabinoid plant extract. The cannabis plant has many naturally occurring substances that are of medical interest. Isolated compounds from the cannabis plant include 19-tetrahydrocannabinol (THC), cannabidiol (CBD), cannabichromene (CBC), cannabigerol (CBG), and cannabidivarin (CBDV), among other compounds. There are at least one hundred and forty one (141) cannabinoids that have been isolated from the cannabis plant.

Cannabinoids

Cannabinoids are a class of chemical compounds that acts on cannabinoid receptors, and include compounds found in cannabis, endogenous compounds, and synthetic cannabinoids. Examples of cannabinoids include, but are not limited to, cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) and tetrahydrocannabinol (THC). Cannabinoids described in the present application include, but are not limited to, those listed in Table 1 below along with their standard abbreviations and chemical structure.

TABLE 1

| | | |
|---|---|---|
| CBD | Cannabidiol | 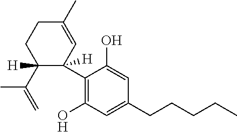 [SMILES: C=C(C)[C@@H]1CCC(C)=C[C@H]1c1c(O)cc(CCCCC)cc1O] |
| CBDA | Cannabidiolic acid | 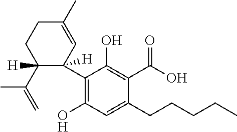 [SMILES: C=C(C)[C@@H]1CCC(C)=C[C@H]1c1c(O)cc(CCCCC)c(C(=O)O)c1O] |
| CBDV | Cannabi- divarin | 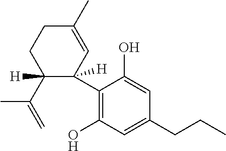 [SMILES: C=C(C)[C@@H]1CCC(C)=C[C@H]1c1c(O)cc(CCC)cc1O] |
| CBDVA | Cannabi- divarinic acid | 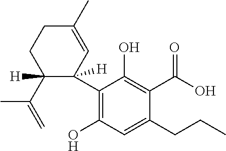 [SMILES: C=C(C)[C@@H]1CCC(C)=C[C@H]1c1c(O)cc(CCC)c(C(=O)O)c1O] |
| THC | Tetrahydro- cannabinol | 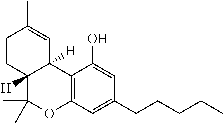 [SMILES: CCCCCc1cc(O)c2c(c1)OC(C)(C)[C@@H]1CCC(C)=C[C@@H]21] |

In embodiments, the chewing gum composition comprises CBD, another cannabinoid, combinations of CBD and other cannabinoids, or combinations of other cannabinoids. In embodiments, the CBD or other cannabinoid is in the form of a highly purified extract of cannabis such that the CBD or another cannabinoid is present at greater than 98% of the total extract (w/w). In embodiments, the cannabinoid tetrahydrocannabinol (THC) has been substantially removed, to a level of not more than 0.15% (w/w) and/or the propyl analogue of CBD, cannabidivarin, (CBDV) is present in amounts of up to 1%. In embodiments, the cannabis extract comprises less than about 0.10%, 0.05%, or 0.01% THC (w/w). In embodiments, the highly purified extract comprises no more than 0.15% CBDA and/or no more than 0.5% CBD-C4. Alternatively, the CBD may be a synthetically produced CBD.

In embodiments, the cannabinoid used in the chewing gum composition is obtained as a liquid carbon dioxide extract of high-CBD containing varieties of Cannabis sativa L. which had been further purified by a solvent crystallization method to yield CBD. The crystallization process specifically removes other cannabinoids and plant components to yield greater than 98% CBD. Although the CBD is highly purified, because it is produced from a cannabis plant rather than synthetically, there are other cannabinoids which are co-produced and co-extracted with the CBD. Similar methods for extracting cannabinoids other than CBD are also known in the art.

In embodiments, the chewing gum composition comprises about 2 to about 50 or more mg of a cannabis extract. In embodiments, the chewing gum composition comprises about 0.1 to about 50 or more mg of one or more cannabinoids. In embodiments, the chewing gum composition comprises about 0.1 to about 90 mg, or more, of CBD, for example, about 0.1 mg to about 5 mg, about 1 mg to about 10 mg, about 5 mg to about 15 mg, about 10 mg to about 20 mg, about 15 mg to about 25 mg, about 20 mg to about 30 mg, about 25 mg to about 35 mg, about 30 mg to about 40 mg, about 35 mg to about 45 mg, about 40 mg to about 50 mg, about 45 mg to about 55 mg, about 50 mg to about 60 mg, about 55 mg to about 65 mg, about 60 mg to about 70 mg, about 65 mg to about 75 mg, about 70 mg to about 80 mg, about 75 mg to about 85 mg, or about 80 mg to about 90 mg, or more.

In embodiments, the chewing gum composition comprises CBD and THC. In such embodiments, the CBD is present in about 0.1-50 mg or more and the THC is present in about 0.1-50 mg or more, for example CBD and THC are independently present in about 0.1 mg to about 10 mg, about 1 mg to about 10 mg, about 5 mg to about 15 mg, about 10 mg to about 20 mg, about 15 mg to about 25 mg, about 20 mg to about 30 mg, about 25 mg to about 35 mg, about 30 mg to about 40 mg, about 35 mg to about 45 mg, or about 40 mg to about 50 mg.

Preparation of Herbal Extracts

The compositions and methods described herein employ herbal extracts that are prepared from the corresponding plant material. Black cohosh extract, for example, can be prepared from black cohosh by the following procedure. First, the raw black cohosh plant material is harvested and dried, for example, by sun-drying or applying heat. Next, the dried black cohosh material is minced, followed by extraction in an aqueous/organic solvent mixture. An aqueous phase is used to extract the water-soluble black cohosh active ingredients, while the organic solvent is used to extract the water-insoluble black cohosh components. The organic solvent can be, for example, one or more of alcohol (e.g., ethanol), chloroform, acetone or ethyl acetate. The amount of solvent in the water can be between 0.1% and 99.9%, for example, about 60% ethanol in water.

The black cohosh active ingredients are extracted by exposing the black cohosh plant material to the water:solvent mix. The exposure time is indirectly proportional to the temperature of the mixture. The temperature of the mix can range between room temperature and boiling temperature. The exposure time can be between about 1 hour to 4 weeks. Times for optimal recovery of active ingredients include, for example, 2 weeks at 50° C. or 24 hours at 90° C. The supernatant is separated from the undissolved plant material, and the organic solvent removed by distillation. Finally, the aqueous supernatant is concentrated, usually, for example, distillation. Similar extraction protocols may be used to obtain wild yam and damiana leaf extracts.

Methods of Treating the Pain and Discomfort Associated with Menstruation

The chewing gum compositions described herein are used in a method for treating the pain and discomfort associated with menstruation including include abdominal pain, back pain, pelvic pain, abdominal distension, diarrhea, and constipation. Treatment of pain and discomfort associated with menstruation is accomplished by administering a chewing gum composition disclosed herein to a subject in need thereof. The term treatment is used in its broadest sense and, as used herein, means lessening the severity, or otherwise alleviating the pain and discomfort associated with menstruation. Administering as used herein means directing to take or taking (e.g., placing in the mouth and chewing) the chewing gum compositions described herein.

In embodiments, the chewing gum for the treatment of pain and discomfort associated with menstruation comprises a therapeutically effective amount of one or more herbal extracts comprising black cohosh extract, wild yam extract and/or damiana leaf extract. During the chewing process, the active ingredients in the herbal extract directly enter the blood stream through the oral mucosa (buccal or sublingual) allowing for fast alleviation of menopausal symptoms, such as hot flashes and night sweats. In embodiments, the herbal extract comprises black cohosh, damiana leaf and wild yam extracts. The herbal extract absorption through the oral mucosa is up to five times faster than conventional ingestible orally delivered products such as capsules, pills or liquids because it bypasses the digestive system and directly enters the bloodstream.

The present disclosure provides a chewing gum delivery system that provides a therapeutically effective concentration of the active agents derived from the herbal extract in the bloodstream within five minutes, and preferably within 1-2 minutes, of administration. In certain embodiments, the chewing gum composition has a high initial release rate of the active ingredients from the herbal extract to provide fast relief of the symptoms of menopause.

The chewing gum composition described provides an herbal extract in a transmucosally absorbable form. Absorption from the gum formulation occurs primarily through the buccal mucosa, which increases the rate of absorption of the active ingredients. The herbal extract administered in the chewing gum formulation are absorbed at a significantly faster rate, while bioavailability is comparable to, or greater than, that of the capsule formulation. As the onset of action for the gum delivery is within 5 to 10 minutes, or less, of administration, the dose of herbal extract can be quickly and easily titrated. Consequently the chewing gum composition described is a convenient and effective means of rapidly administering an herbal extract, and is useful for the rapid alleviation of discomforting symptoms associated with menstruation.

The method of treating the pain and discomfort associated with menstruation comprise chewing a piece of gum comprising an herbal extract as described herein, following the onset of pain or discomfort associated with menstruation. Typically, up to two pieces of the chewing gum composition may be chewed at one time. If symptoms associated with menstruation continue after chewing of 1-2 pieces of gum, an additional piece of gum may be chewed.

The chewing gum compositions disclosed herein may be administered prophylactically to prevent pain or discomfort associated with menstruation. The compositions disclosed herein may be administered alone or may be administered in combination with one or more other therapeutic agents.

Manufacture of the Gum Compositions

The chewing gum compositions described herein comprise a gum base and an herbal extract including black cohosh extract, damiana leaf extract and/or wild yam extract. In embodiments, methods for the preparation of the chewing gum composition comprising the herbal extract avoid heating and cooling of the active ingredient.

In an embodiment, the method for manufacture of the chewing gum is exemplified in U.S. Pat. No. 9,744,128, incorporated herein by reference. In general the process for preparing the chewing gum compositions comprises placing one or more gum base(s) in a container with a sugar alcohol and heating the gum base(s) and one or more sugar alcohols in an oven to melt the gum base(s) to an internally-measured temperature between 140-160° F. to melt the gum base(s). The gum base may contain one or more elastomers, resins, softening agents, emulsifiers and/or fillers. The ingredients, including one or more active ingredients, sugar alcohol(s) and flavorings are then combined and mixed in a commercial mixer. The melted gum base is added to the mixer and mixed until a homogenous mass is produced, and cooled to produce a particulate mixture. The temperature of the gum base initially exceeds that of the mixer when first introduced, but as mixing continues the mixture cools to room temperature and forms granular pieces. These granular pieces are then conditioned for a period of time, which allows the granular pieces to dry slightly and complete the crystallization process. The pieces are conditioned for at least about 6 hours at a temperature not greater than about 75° F. and about 60% or less relative humidity. The dried mass is then milled into particulates at room temperature using a mesh screen of appropriate size. The particulates are mixed with tableting excipients (e.g., one or more lubricants/glidants) in an orbital or planetary mixer, and tableted using a tablet press. This process preserves the efficacy of the active ingredient or ingredients by avoiding exposure to extreme temperatures, particularly during the mixing and milling.

In embodiments, other gum base compositions may utilized in the chewing gum compositions disclosed herein. The gum base can be prepared via application of traditional mixing that utilizes mechanical actions while heating the mixture.

In an embodiment, a mixing pot is preheated to approximately 115-125° C. After about 10 minutes of preheating the elastomer, filler and additional softening ingredients are added to a mixing pot. The rubber along with the resin is mixed until the mixture is consistent. The softening ingredients can be added after 30-50 minutes and mixed with the rubber and resin to form a consistent mixture. The mixture is then released into another pot to cool down to approximately 18-25° C.

In an embodiment, the mixing pot is preheated to approximately 55° C. After 10 minutes of preheating the gum base and filler are mixed in the mixing pot. After the mixture is consistent, additional ingredients are added. The herbal extract is added in the first half of the mixing process.

Chewing Gum Compositions

The present disclosure provides a chewing gum composition comprising a gum base and an herbal extract along with additional excipients. In embodiments, the composition comprises black cohosh extract and a gum base. In some embodiments the composition further comprises damiana leaf extract and/or wild yam extract. The chewing gum compositions can additionally include a sugar, a sugar blend, a sugar alcohol, a blend of sugar alcohols or combinations thereof, as well as additional excipients such as coloring agents, flavoring agents, lubricants and powder flow agents. In further embodiments the composition comprises one or more sugar substitutes and/or flavoring agents.

In embodiments, the chewing gum composition comprises one or more sugar alcohols, a gum base, one or more flavoring agents, one or more sweeteners, an herbal extract, and tableting excipients. In further embodiments, the composition comprises about 45-65% sugar alcohols, about 15-35% gum base, about 2-15% flavoring agents and/or sweeteners, about 2-10% herbal extract and about 1-10% tableting excipients. In embodiments, the composition comprises about 55% sugar alcohols, about 27% gum base, about 9% flavoring agents and/or sweeteners, about 5% herbal extract and about 3% tableting excipients. In embodiments, the herbal extract is selected from black cohosh, damiana leaf and/or wild yam.

In an embodiment, the chewing gum composition comprises about 5% to about 80% of a gum base; about 10% to about 80% by weight of a sugar, a sugar blend, sugar alcohol, blend of sugar alcohols, sweetener, or combinations thereof; about 1% to about 20% by weight of a flavoring agent; about 0.1% to about 10% by weight of a lubricant or powder flow agent, or combinations thereof. In an embodiment, the chewing gum composition comprises about 55% to about 75% of a sugar, a sugar blend, sugar alcohol, blend of sugar alcohols, sweetener, or combinations thereof, about 20% to about 30% of a gum base, about 1% to about 15% of a flavoring agent or agents, about 1% to about 20% of an herbal extract, about 0.1% to about 5% tableting lubricants and powder flow agents; and about 0.01% to about 2% by weight of one or more sugar substitutes.

Gum Base

The gum base imparts the chewing characteristics to the final gum composition. The gum base can also impact the release profile of the active ingredients, flavors and sweeteners. A suitable chewing gum base for use in the gum compositions described herein comprises one or more constituents including one or more elastomer, resin, plasticizer or softener, and filler. Certain components in the gum base may have more than one function in the composition. The gum base may also contain one or more stabilizing agents, coloring agents, and flavoring agents. In a preferred embodiment, the gum base comprises Couma macrocarpa, glycerol esters of gum, microcrystalline wax, lecithin, and calcium carbonate.

In embodiments, the chewing gum composition comprising the herbal extract comprises an elastomeric or natural chicle base as is commonly used in chewing gum formulations that are commercially available and accepted by the consumer. An elastomeric or natural chicle base is present in the chewing gum composition in an amount of about 10% to about 85% by weight, based on the total weight of the chewing gum composition.

Elastomers provide the rubbery, cohesive nature to the gum and may include natural or synthetic types. The elastomer may be any water-insoluble polymer including the natural and synthetic gum polymers utilized for chewing gums listed in the U.S. Code of Federal Regulations, Title 21, Section 172.615 (incorporated herein by reference). Useful natural elastomers include natural rubber such as smoked or liquid latex and guayule, and natural gums such as jelutong, couma macrocarpal (also called lechi caspi), perillo, sorva, massaranduba balata, massaranduba chocolate, nispero, rosidinha, chicle, gutta percha, gutta kataiu, niger gutta, tunu, chilte, chiquibul, and gutta hang kang.

Synthetic elastomers include high-molecular weight elastomers such as butadiene-styrene copolymers, polyisobutadiene and isobutylene-isoprene copolymers, low-molecular weight elastomers such as polybutene, polybutadiene and polyisobutylene, vinyl polymeric elastomers such as polyvinyl acetate, polyethylene, vinyl copolymeric elastomers such as vinyl acetate/vinyl laurate, vinyl acetate/vinyl stearate, ethylene/vinyl acetate, polyvinyl alcohol, or mixtures thereof. Combinations of synthetic elastomer in the gum base can include a synthetic elastomer having a high-molecular weight and a low-molecular-weight elastomer. Combinations of synthetic elastomers include, but are not limited to, polyisobutylene and styrene-butadiene, polyisobutylene and polyisoprene, polyisobutylene and isobutylene-isoprene copolymer (butyl rubber) and a combination of polyisobutylene, styrene-butadiene copolymer and isobutylene isoprene copolymer, and all of the above individual synthetic polymers in admixture with polyvinyl acetate, vinyl acetate-vinyl laurate copolymers, respectively and mixtures thereof.

Resins provide a cohesive body or strength to the gum composition and may also act as softeners and include glycerol esters of gum, terpene resins, and/or polyvinyl acetate.

Plasticizers (softening agents) affect the firmness of the gum base. Elastomer plasticizers include natural rosin esters often referred to as ester gums. Such plasticizers include methyl, glycerol and pentaerythritol esters of rosins and modified rosins, such as hydrogenated, dimerized and polymerized rosins. Examples are glycerol ester of wood and gum rosin, glycerol ester of partially hydrogenated wood and gum rosin, glycerol ester of polymerized wood and gum rosin, glycerol ester of partially dimerized wood and gum rosin, glycerol ester of tall oil rosin, pentaerythritol ester of wood and gum rosin, pentaerythritol esters of partially and fully hydrogenated wood and gum rosin, methyl esters of wood and gum rosins and partially and fully hydrogenated methyl esters of wood and gum rosin. Synthetic plasticizers include terpene resins derived from α-pinene, β-pinene and/or dipentene.

Emulsifying agents may act as softeners and can also help to hydrate the composition. In embodiments, the emulsifier is lecithin and/or glycerol monostearate. Emulsifiers include monoglycerides, diglycerides, acetylated mono and diglycerides, distilled mono- and diglycerides, glycerol monostearate, propylene glycol monostearate, Na-, K-, Mg- and Ca-stearates, glycerol triacetate, fatty acid monoglycerides (e.g., stearic, palmitic, oleic and linoleic acids), lactic acid esters and acetic acid esters of mono- and diglycerides, sugar esters of edible fatty acids, lecithin and hydroxylated lecithin.

One or more fillers may be used in the compositions disclosed herein to modify the texture and aid in processing. In embodiments, the filler comprises talc and/or calcium carbonate. Fillers include celluloses and cellulose derivatives including microcrystalline cellulose, hydroxypropylcellulose and sodium carboxymethylcellulose, lactose, starches including potato starch and corn starch, carbohydrates including a cellulose derivative, e.g. hemicellulose. The cellulose derivative may be of natural origin, e.g. dextran, agarose, agar, pectin, alginate, xanthan, chitosan, starch. The cellulose derivative may also be of synthetic or semi-synthetic origin. In certain embodiments, the filler comprises microcrystalline cellulose (MCC), bamboo fibers, or combinations thereof. Specific examples of a suitable microcrystalline cellulose is microcrystalline cellulose comprising: AVICEL™ grades PH-100, PH-102, PH-103, PH-105, PH-112, PH-113, PH-200, PH-300, PH-302, VIVACEL™ grades 101, 102, 12, 20 and EMOCEL™ grades 50M and 90M, and the like, and mixtures thereof. The fillers may be present in the composition from about 5% to about 50% by weight, based on total weight of the composition.

Sweeteners

Sweeteners may be used in the compositions disclosed herein to impart a sweet taste to the gum composition. In addition, sweeteners can act a softener and/or as a filler in the gum composition. Suitable sweeteners include one or more of a sugar, sugar blend, sugar alcohol, a blend of sugar alcohols, high intensity sweeteners, sugar substitutes, or combinations thereof. Sugar substitutes include sucralose, monk fruit, neotame, licorice extract, stevia, honey or agave nectar, or combinations thereof. Suitable high intensity sweeteners include sucralose, stevia, honey, monk fruit, neotame, licorice extract, agave nectar, or combinations thereof. Sugar alcohols include sorbitol, isomalt, xylitol, maltitol, mannitol, erythritol or combinations thereof. Sugars include dextrose, sucrose, fructose, glucose or combinations thereof. The amount of sweetener in the gum composition depends on factors such as potency of the sweetener, rate of release, desired sweetness of the product, and amount and type of flavor used.

Flavorings

Natural and/or artificial flavorings may be used in the gum compositions disclosed herein. Both synthetic flavoring agents and natural flavoring agents derived from plants, leaves, flowers, fruits, etc. and combinations thereof can be used. Examples of flavoring agents include spearmint oil, cinnamon oil, oil of wintergreen (methysalicylate) and peppermint oils. Synthetic and natural fruit flavors useful as flavoring agents include citrus oil, e.g., lemon, orange, lime and grapefruit; fruit essences including apple, strawberry, cherry, banana, pineapple; and other flavorings such as aldehydes and esters including cinnamyl acetate, cinnamaldehyde, citral diethyl acetal, dihydrocavryl acetate, eugenyl formate, p-methylamisol, and others. Flavoring agents are generally liquids. However, they can also be used as spray dried solids. The use of flavoring agents having other physical forms such as powdered flavorings, beaded flavorings and encapsulated flavorings may also be used in the gum compositions disclosed herein. In embodiments, the flavoring agent or agents in the chewing gum composition is mint, peppermint, citrus, mixed berries, spearmint, wintergreen, and combinations thereof

The amount of flavoring agent employed is determined by flavor type and the strength of flavor desired. In embodiments, flavoring amounts of about 0.05% to about 3.0% by weight of the overall chewing gum composition are used, preferably about 0.3% to about 1.5%, more preferably about 0.7% to about 1.2% by weight.

In embodiments, additional flavoring agents are added to the chewing gum composition granules immediately prior to tableting. Preferably, the additional flavoring agent is in a dry form, e.g., spray dried flavoring or encapsulated flavoring. However, liquid flavoring can be added if it is first blended with the dry components of the lubricating system.

Lubricants and Powder Flow Agents

Lubricants and powder flow agents (e.g., glidants) may be used in the compositions of the present disclosure to aid processing. Tableting lubricants and powder flow agents include magnesium stearate, silicon dioxide, calcium stearate, stearic acid, talc, rice bran-based excipients, or combinations thereof.

Binders

Binders include water-soluble synthetic polymer, polyvinlypyrrolidone (povidone), sorbitol, mannitol, xylitol, lactitol, erythritol, pregelatinized starch, modified starch (e.g., starch sodium octenylsuccinate), hydroxypropylmethylcellulose and others.

Stabilizing Agents

In embodiments, the gum compositions contain one or more stabilizing agent to prolong the shelf life of the active ingredient(s) and/or other components of the gum composition. In embodiments, the stabilizing agent is an antioxidant. Suitable antioxidants include butylated hydroxyanisole (BHA), butylated hydroxytoluene (BHT), betacarotenes, tocopherols, acidulants such as vitamin C, propyl gallate, and combinations thereof.

All references referred to herein are incorporated in their entirety.

### Examples

Preparation of the Chewing Gum Containing an Herbal Extract: In general the process for preparing the chewing gum compositions comprises placing the components of the gum base in a container with a sugar alcohol and heating in an oven to an internally-measured temperature between 140-160° F. to melt the gum base. The ingredients, including the active ingredients (from black cohosh extract, damiana leaf extract and wild yam extract), sugar alcohol(s) and flavorings are then combined and mixed in a commercial mixer. The melted gum base is added to the mixer and mixed until a homogenous mass is produced, and cooled to produce a particulate mixture. The temperature of the gum base initially exceeds that of the mixer when first introduced, but as mixing continues the mixture cools to room temperature and forms granular pieces. These granular pieces are then conditioned for a period of time, which allows the granular pieces to dry slightly and complete the crystallization process. The pieces are conditioned for at least about 6 hours at a temperature not greater than about 75° F. and about 60% or less relative humidity. The dried mass is then milled into particulates at room temperature using a mesh screen of appropriate size. The particulates are mixed with tableting excipients in an orbital or planetary mixer, and tableted.

TABLE 2
Formulation of chewing gum

| Ingredient | wt % in gum | Function |
|---|---|---|
| Herbal extract | 5.4 | Active agents |
| black cohosh extract | | |
| damiana leaf extract | | |
| wild yam extract | | |
| Gum Base | 27.3 | elastomer |
| couma macrocarpa | | resin |
| glycerol ester of gum | | softening agent |
| microcrystalline wax | | emulsifier |
| lecithin | | filler |
| calcium carbonate | | |
| Sugar Alcohol | 55.5 | sweetener |
| maltitol | | |
| sorbitol | | |
| isomalt | | |
| xylitol | | |
| Flavoring agents and | 8.7 | Flavoring agent, |
| sweeteners (stevia extract, | | sweetener |
| mint flavoring) | | |
| Tableting excipients (silicon | 3.1 | Glidant, lubricant |
| dioxide, Mg stearate) | | |

1. A composition comprising a black cohosh extract and a gum base.

2. The composition of claim 1, wherein the composition further comprises damiana leaf extract and/or wild yam extract.

3. The composition according claim 1, wherein the gum base comprises Couma macrocarpa, glycerol esters of gum, microcrystalline wax, lecithin and calcium carbonate.

4. The composition according to claim 1, further comprising one or more sugar alcohols.

5. The composition of claim 4, wherein the one or more sugar alcohols comprises one or more of maltitol, sorbitol, isomalt, and xylitol.

6. The composition according to claim 1, further comprising one or more additional sweeteners.

7. The composition of claim 6, wherein the one or more additional sweeteners are selected from a sugar, a high intensity sweetener, a sugar substitute, or a combination thereof.

8. The composition according to claim 1, further comprising one or more flavoring agents, and one or more lubricants and powder flow agents.

9. The composition according to claim 1, wherein the composition comprises about 45-65% sugar alcohols, about 15-35% gum base, about 2-15% flavoring agents and/or sweeteners, about 2-10% herbal extract and about 1-10% tableting excipients.

10. The composition of claim 9, wherein the composition comprises about 55% sugar alcohols, about 27% gum base, about 9% flavoring agents and/or sweeteners, about 5% herbal extract and about 3% tableting excipients.

11. The composition according to claim 1, further comprising maltitol, sorbitol, isomalt, xylitol, Stevia, a flavoring agent, magnesium stearate, and silicon dioxide.

12. The composition according to claim 1, further comprising one or more of chromium picolinate, calcium disuccinate, ammonium succinate, zinc difumarate hydrate, ocopherol acetate, glycine, triterpene glycosides, folic acid, vitamin B12, vitamin E, vitamin B6, riboflavin and selenium.

13. The composition according to claim 1, further comprising one or more cannabinoids.

14. The composition of claim 13, wherein the one or more cannabinoids comprises a cannabinoid selected from the group consisting of cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM) and combinations thereof.

15. The composition of claim 14, wherein the one or more cannabinoids is CBD.

16. A method of treating symptoms associated with menstruation comprising: administering to a subject in need thereof, a composition according to claim 1.

17. The method of claim 16, wherein the one or more symptoms associated with menstruation are selected from the group consisting of abdominal pain, back pain, pelvic pain, abdominal distension, diarrhea, and constipation.

18. The composition of claim 1, wherein the composition is obtained by a method comprising the steps of:
(i) placing the gum base in a container with a sugar alcohol and heating to an internally-measured temperature of between 140-160° F. to melt the gum base;
(ii) combining the herbal extract, sweeteners and flavoring agents, and mixing;
(iii) adding the melted gum base mixture to the mixture from step (ii) and mixing;
(iv) conditioning the mixture from step (iii) for at least about 6 hours at a temperature less than or equal to 75° F. and a relative humidity of 60%;
(v) milling to form particulates;
(vi) mixing the particulates of step (v) with a lubricant; and
(vii) tableting the mixture from step (vi).
